(19) United States
(12) Patent Application Publication
(10) Pub. No.: US 2006/0275753 A1
(43) Pub. Date: Dec. 7, 2006

(54) Recovery of analytes using combinatorial libraries

(76) Inventors: David J. Hammond, Laytonsville, MD (US); Julia Tait Lathrop, Falls Church, VA (US)

Correspondence Address: MERCHANT & GOULD PC, P.O. BOX 2903, MINNEAPOLIS, MN, 55402-0903, US

(21) Appl. No.: 11/454,799

(22) Filed: Jun. 19, 2006

Related U.S. Application Data
(63) Continuation-in-part of application No. 10/414,523, filed on Apr. 14, 2003.
(63) Continuation-in-part of application No. 10/601,032, filed on Jun. 20, 2003.
(60) Provisional application No. 60/372,091, filed on Apr. 15, 2002.
(60) Provisional application No. 60/395,038, filed on Jul. 11, 2002.

(51) Int. Cl. C12Q 1/70; C12Q 1/68; G01N 33/53; G01N 33/92; G01N 33/00

(52) U.S. Cl. 435/5; 435/7.1; 435/6; 436/86; 204/450; 436/71

(57) ABSTRACT

The invention provides a method of binding multiple targets in samples by binding to multiple ligands. The method comprises providing ligands attached to a support, and contacting the ligands with targets to produce at least two target-ligand-support complexes. The method further comprises removal of non-bound targets followed by elution of the bound targets. The eluted targets are present in concentrations of a particular analyte that is a function of their comparative concentrations in different samples. Furthermore, the mixture is enriched for trace components.

### CROSS REFERENCE TO RELATED CASES

This application is a continuation-in-part of U.S. patent application Ser. No. 10/414,523, filed Apr. 14, 2003, which claims priority to U.S. Provisional Application Serial No. 60/372,091, filed Apr. 15, 2002, the contents of each of which are incorporated by reference herein in their entirety. This application is also a continuation-in-part of U.S. Patent Application Serial No. 10/601,032, filed Jun. 20, 2003, which claims priority to U.S. Provisional Application Serial No. 60/395,038, filed Jul. 11, 2002, the contents of each of which are also incorporated by reference herein in their entirety.

### BACKGROUND

1. Field of the Invention

The field relates to sample preparation devices for the improved detection of targets, in which the amount of a target bound to and eluted from the library reflects its initial concentration in one sample as compared with a second, comparable sample, such that a greater amount of target will be detected from a sample with an initial higher concentration than a second, comparable sample with a lower initial target concentration, for improved diagnostics, and biomarker discovery.

2. Background of the Invention

Proteomics seeks to identify and characterize multiple proteins simultaneously. Investigation of whole blood is highly desirable, but is complicated by having multiple proteomes, e.g. red blood cells, platelets, macrophages and plasma. Thus, blood is usually fractionated into component fractions prior to analysis. Blood plasma and serum fractions, comprising the largest and deepest version of the human proteome spanning 10¹⁰ or more orders of magnitude of concentration of individual targets, are the primary specimens for the analysis of existing biomarkers and diagnostic indicators of disease, and for the discovery of new biomarkers and diagnostics. The number of proteins present in plasma and serum is immense, particularly when considering, post-translational micro- heterogeneity, variations in glycosylation and other post-translational modifications, proteolytic fragmentation, protein-protein complexes and the antibody repertoire, which alone may comprise 10,000,000 different proteins. These attributes make plasma and serum particularly difficult to analyze, and prevent most analysis of whole blood.

The enormous depth in concentration and complexity reflects the dynamic range (difference between the highest and lowest concentration) (Lathrop, J. T., Carrick, K., Hayes, T. K., Hammond, D. J. (2005) “Rarity Holds a Charm”: Evaluation of trace proteins in plasma and serum (invited review). Expert Review of Proteomics 2(3):393-406) and Anderson (J. Physiol 563.1 2005 pp23-60, Topical review Candidate-based proteomics in the search for biomarkers of cardiovascular disease), Anderson N. L. and Anderson, N. G, (“The human plasma proteome, History, Character and diagnostic prospects,” Molecular and Cellular Proteomics 1(11): 845-867 (2002)) with albumin at about 40 mg/ml and cytokines such as IL-6 at ˜1-10 pg/ml or lower; however, current technology, i.e. mass spectrometry and 2-dimensional gel electrophoresis is limited to a dynamic range of 10³-10⁴. Consequently, to identify trace components it is necessary to selectively enrich trace components over many orders of magnitude relative to the abundant proteins.

There are a number of approaches for selective enrichment of trace entities, especially proteins, most of which involve separation or removal of the entities present at the highest concentrations. Proteins may be digested by proteases, especially trypsin. The resultant peptides may then be fractionated by multi-dimensional chromatography prior to analysis by tandem mass spectrometry, i.e. MudPIT. Alternatively, the proteins themselves may be fractionated by chromatography, e.g. ion exchange, reverse phase, metal chelate, gel filtration, and protein-specific or group-specific affinity separation prior to analysis.

An alternative approach is to selectively deplete the abundant proteins with specific monoclonal antibody affinity columns. Selective depletion strategies are most often targeted to albumin, IgG, IgA, transferrin, haptoglobin, alpha-i proteinase inhibitor (API) also known as antitrypsin, and fibrinogen. These monoclonal affinity columns are expensive and seldom totally specific for their target; furthermore, they only decrease the concentration range by a couple of orders of magnitude, leaving the trace proteins still below the limits of detection and still masked by the next set of most abundant proteins.

Thus, the above strategies will separate major abundant species from trace components, but all have significant disadvantages including making the sample unstable because of the absence of the abundant proteins and further diluting trace components.. Moreover, many trace targets bind to the abundant species, especially albumin, antibodies, fibrinogen and alpha-2 macroglobulin, and thereby may be even further depleted during fractionation. Furthermore, the methodologies, especially monoclonal antibody-based depletion, are specific for the proteins in an individual tissue from a single, or closely related, species. Finally, the immunoglobulins, which are among the most valuable class of proteins as biomarkers of infection and as therapeutics, are frequently removed and generally are not amenable to evaluation.

Applicants' priority application, Hammond and Lathrop (U.S. patent application Ser. No. 10/414,523, “Method for detecting ligands and proteins in a mixture”) teaches a technology now termed the “Bead Blot” that uses combinatorial libraries to bind representative amounts of most, if not all, the proteins present in a sample on an inert support and transfers these proteins under one or more conditions to a second support such as a membrane. Since all the components within a sample can be captured in unique positions on a second matrix such as the membrane, the second matrix can be screened sequentially or simultaneously for the presence of multiple, independent targets. This technology was designed, in part, to identify proteins associated with a diseased state.

The Bead Blot also can be used for quantifying the amount of target in a sample, detecting differences in proteins expressed from cells under different conditions, or in separating and detecting proteins present in biological samples (e.g. plasma or other biological fluid) associated with a disease state versus a normal state.

Alternatively, instead of being placed in a matrix and the targets eluted from them, the combinatorial beads with the representative amounts of bound entities can be subdivided and evaluated for a desired chemical, e.g. by mass spectrum, and chemical composition; biochemical property, e.g. enzyme activity or interaction property, or biological activity, e.g. cell growth, death or differentiation (Hammond D J, Lathrop J T, Sarkar J, Gheorghiu, L. WO 2004/007757). The desired activity optionally can be directly traced back to the individual bead, or sub-pool of beads from which it was selectively bound.

An additional patent employing combinatorial technology for sample preparation (Boschetti and Hammond, “Methods for reducing the range in concentrations of analyte species in a sample,” U.S. patent application Ser. No. 11/089,128) relates specifically to the compression of the analyte concentration range by decreasing the concentration range between a number of different analytes in the original sample. This was achieved using the combinatorial libraries described in the Bead Blot patent as the binding moieties and evaluating the bound and then eluted targets en masse or in sub-pools as described in WO 2004/007757, or following elution by different sequential or parallel elution conditions as described in U.S. patent application Ser. No. 10/414,523.

Boschetti and Hammond state “at one extreme, the relative amount of binding moieties to analytes may be so large that the binding moieties are able to capture all of the analytes in the sample. In this case, there is no compression of the analyte concentration range. At the other extreme, the relative amount of binding moieties to analytes may be so small, that every analyte species saturates the ability of the binding moieties to bind. In this case, theoretically, the amount of each analyte species captured is the same, and the range in analyte concentration is compressed to equality. This extreme is particularly useful when the goal is to detect as many species as possible. Between these two extremes is the situation in which the more abundant species saturate the binding moieties, while the less abundant species do not saturate the binding moieties. In this case, there is little difference in concentration of the abundant analytes, while differences in concentration of the less abundant species remain.”

Thus, a major problem with the conceptual approach of “Equalization” is the necessity to operate within a specific concentration range of analytes to ligands. Moreover, in biomarker discovery it is important to measure the relative concentration as well as the presence of an analyte. For example, patients with a steady state CRP level above 3 mg/L are considered to be at risk for cardiovascular disease while those with higher, but decreasing levels may be recovering from a natural, and desirable, acute phase reaction to a pathogen. Those at levels below 1 mg/L are considered to be normal. In addition, increasing levels of a biomarker over time may indicate progressive tissue failure, e.g., increased troponin levels may indicate a myocardial infarction, while static, but elevated, levels may indicate a chronic disease state. Furthermore, the discovery of new biomarkers will not be restricted to a single concentration range of targets; thus it is necessary to preserve the relative concentrations between samples of the trace, intermediate and more abundant analytes.

Thus, there remains a major unmet need to concentrate trace components from complex mixtures in a manner that reflects the original analyte concentration in one sample versus a second sample, preserving the concentration differentials of the analytes between samples. The amount of a particular analyte that is bound is dependent on, among other parameters, its original concentration, the number of ligands present in the library to which it binds, and the range of affinities and specificities of these ligand: target interactions.

### SUMMARY OF THE INVENTION

This invention provides a significant improvement to the identification of analytes (targets) in a complex sample by a) compressing a range of protein concentrations between highly abundant and trace species and b) maintaining the relative differences in concentrations of any given analyte (target) in one sample relative to the amount in a second, comparable sample (by similar or comparable sample it is meant a sample obtained from the same type of source, i.e., blood, urine, saliva, etc.).

The ability to detect different relative amounts of a given analyte depends, in part, upon the complexity of the sample in which it is present. Frequently, it is desirable to identify and quantify plasma proteins, which are present in whole blood, for proteome research and diagnostics; however, the complexity of whole blood and the dynamic concentration range of proteins make it extremely difficult to identify proteins over a concentration range of more than about 104 of the anticipated dynamic range of >10¹⁰ at one time. In addition, the huge complexity resulting from the number of different proteins and their post-translational modifications present enormous analytical limitations.

This invention provides a diverse combinatorial library of ligands that is designed to bind components of a complex sample such as whole blood and plasma. To achieve this, the ligands must present a broad range of equilibrium dissociation constants. This is achieved by providing a library of sufficient diversity, i.e. 1,000 or more ligands.

Briefly, the matrix, e.g. blood which contains red blood cells, white cells, platelets and plasma proteins, is mixed with a combinatorial library of ligands optionally in the presence of a second, batch of affinity support(s) targeted to select proteins that bind to a disproportionately large number of ligands in a library (so-called “highly interactive” or “promiscuous” proteins). The proteins are allowed to bind to the ligands of the library and non-bound cells and other entities are removed by washing. The bound proteins are then eluted from the ligands and characterized by physical, chemical, biochemical or biological means. The amount of a particular target that is bound from one sample versus a second and similar sample (by similar or comparable sample it is meant a sample obtained from the same type of source, i.e., blood, urine, saliva, etc.) is partially a function of and reflects its initial concentration within the samples.

### BRIEF DESCRIPTION OF THE DRAWINGS

FIG. 1 Plasma containing both fibrinogen and API was mixed with the combinatorial ligand library. The proteins were eluted by heating resin in LDS sample buffer at 70° C. and the eluate loaded on the gel at the indicated dilutions. Electrophoresed proteins were transferred to a membrane and the proteins detected with immunological methods. Gel I—incubated with anti-fibrinogen antibody. Gel II—incubated with anti-API antibody.

FIG. 2 Detection of Troponin by Western Blot of troponin-spiked and unspiked plasma samples bound to a ligand library

FIG. 3 Detection of Troponin by ELISA of troponin-spiked and unspiked blood bound samples to a ligand library. Samples were evaluated by Bio-Quant ELISA kit.

FIG. 4 Detection of Troponin by ELISA from blood and plasma first spiked with troponin and then treated with the ligand library, compared with blood first treated with the ligand library and then spiked with troponin. Samples were evaluated by Bio-Quant ELISA kit.

FIG. 5. Comparison of plasma incubated in the presence and absence of a ligand specific for fibrinogen. The lanes containing plasma proteins bound to and eluted from library and “sequestered” library demonstrate substantial decreases in fibrinogen chains (arrows) and increases in other proteins that migrate on the gel in positions near fibrinogen in the “sequestered” lane.

FIG. 6. Flow chart of the “sequestered” library incubation procedure.

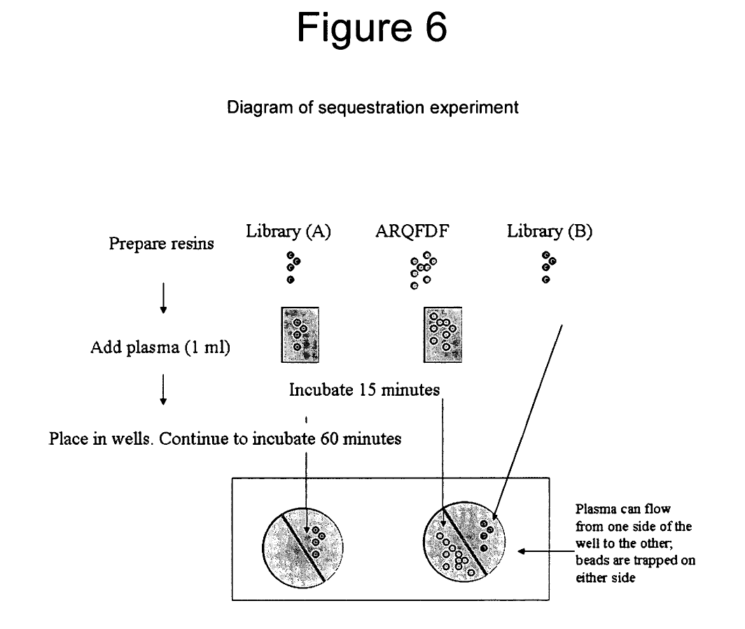

### DETAILED DESCRIPTION OF THE INVENTION

The invention provides a methodology to detect target molecules following separation from a sample and each in an amount that reflects its relative concentration in one starting material compared with its concentration in a second, comparable starting material. In particular, the invention provides a method of evaluating the amount of a target in a sample. The method comprises (i) providing one thousand or more different ligands, wherein each ligand is attached to a support to form one thousand or more ligand-support complexes (ii) contacting the ligand-support complexes with two or more targets in a sample under conditions that allow at least one of the targets to bind to at least one ligand-support complex, thereby forming one or more target-ligand support complexes, (iii) separating bound from non-bound targets, (iv) eluting at least a portion of the target of at least one target-ligand-support complex and (v) detecting the eluted target.

Although the preferred embodiment uses one thousand or more different ligands to produce one thousand or more ligand-support complexes, one of skill in the art could envision the use of fewer ligands. The number of ligands used, of course, will depend on the complexity of the sample being characterized. Although most samples to be characterized are complex enough to require one thousand or more ligand-support complexes, some may only require 900, 800, 700, 600, 500, 400, 300, 200, 100 or fewer. Furthermore, as many as five thousand, ten thousand, fifty thousand, one hundred thousand, five hundred thousand, one million or more different ligand-support complexes can be used in the methods of the claimed invention.

The amount of any particular target that is eluted is related both to the concentration of the target in the original starting material and to the amount of that particular target that is captured by the library, such that the concentration differential is maintained for any one target between the amount that is captured and eluted from one sample relative to the amount of the same target that is captured and eluted from a second comparable sample, in which the target is present at a differential initial concentration.

Alternatively, the method optionally comprises dividing two or more target-ligand-support complexes before step (iv) into sub-pools as described in the parent application, U.S. patent application Ser. No. 10/601,032 then eluting at least a portion of the target of at least two target-ligand-support complexes from one sub-pool in an amount proportional to the amount captured from the starting sample, which is related to its original concentration in the starting sample, and detecting and analyzing the at least one target.

The method also optionally comprises conducting step (iv) in a medium containing a competitive binding agent, which binds to the target of at least one target-ligand-support complex, thereby causing the ligand to dissociate from at least a portion of the target. These competitive binding agents can be a ligand (different from the ligand of the target-ligand-support complex), cofactors for the target, enantiomeric specific molecules, and the like.

The invention also provides a method for reducing the dynamic range of targets in a sample. This method comprises (i) providing a first sample comprising a first plurality of different targets, having a first variance in amounts; (ii) contacting the first sample with a plurality of different binding ligands, each binding ligand present in a determined amount; (iii) binding a portion of the first different targets from the first sample to the different binding ligands and removing unbound targets from the newly formed target-ligand-support complexes; and (iv) eluting the bound targets from the target-ligand-support complex, thereby producing a second sample comprising a second plurality of different targets, whereby the variance in amounts of targets in the second sample is less than the variance in amounts of targets in the first sample.

The invention offers a number of advantages over previous target detection and analysis methods. First, a target can be recovered under conditions which maintain and thus can identify initial differences in levels of concentration of a particular target between different samples. Moreover, in general, trace analytes are preferentially concentrated relative to more abundant species, making the detection of proteins preferentially expressed in a disease state at low levels easier to identify, as well as decreasing interferences from abundant species and increasing the signal-to-noise ratio in assays. In addition, because essentially all the components within a sample can be captured on different ligands immobilized on beads, the beads with bound proteins may be assayed in total either sequentially or simultaneously for the presence of multiple, independent targets, or may be split into a number of different sub-pools or individual beads and evaluated for the binding of targets. The final, total number of targets identified will be the sum of all those identified in the sub-pools. The benefit of summing the components over evaluation of the complete processed samples is that sequential elution of the different sub-pools will produce different target compositions in less complex preparations, thereby making analysis easier.

Yet another advantage of the invention is that the chemical, biochemical, and biological activity of the target can be maintained, if desired by selecting, for example, elution conditions that preserve the targets'conformational structure. Furthermore, elution conditions can be advantageously controlled to transfer a subpopulation of the bound targets at any one time or to identify specific elution conditions of selected targets. Moreover, it is also possible to identify targets that specifically bind to selected molecules by using an elution buffer containing that molecule.

Theoretical explanation

To date there are no mathematical models to describe the binding of a large number of analytes with varied affinities to a large number of ligands. Such modeling would need to consider such variables as:

Target properties- stability, relative concentrations, size/molecular weight, composition, e.g. similarity in structural properties of the targets, overall diversity and concentration range.

Binding conditions- batch versus column format, resin versus membrane, contact time, pH, flow, shear rates, mixing, and temperature.

Binding medium composition- pH, ionic strength, viscosity and temperature.

Ligand properties- chemical composition, diversity and density of the ligands

Support polymer- diameter, porosity, non-specific binding, chemical and biochemical interactions with the sample.

Competition between different analytes for some of the same ligands

Competition for binding of a target to ligands by soluble inhibitors.

Additional binding of the target to targets already associated with target-ligand-support complexes.

Additional binding of the target through its association with binding partners of a multi-enzyme complex.

The stringency and time of washing to remove non-bound analytes.

Elution conditions and the methods employed for detecting the target.

Thus, the amount of any one analyte bound to a combinatorial library of ligands may vary significantly compared with the amount bound of a second analyte. For example, fibrinogen and HDL have a high number of high affinity ligands and are referred to as “most interactive proteins”, perhaps due to such features as stronger and more effective binding through multi-point attachment of multiple ligands on one support to identical subunits within the protein. In addition, different subunits may bind to ligands with different structures on alternative beads, thereby further increasing the number of affinity interactions, because each subunit may have multiple and different interactive binding sites. Furthermore, proteins may exist as part of a protein complex, and binding of the complex may be mediated by any one of several partners in the complex. For example, fibrinogen is comprised of six subunits, and binds many other target proteins, including fibronectin, factor XIII, and von Willebrand factor. The HDL complex contains paraoxonase, apolipoprotein (apo) Al, apo All, apo AIV, apo B100, apo D, apo E and other proteins. In contrast, proteins like transferrin and Alphal Proteinase Inhibitor (API), which have a low number of high affinity ligands, are monomers with very few binding interactions and sites.

Furthermore, there is a gradation of the relative amounts of any one protein that will bind to individual supports bearing a ligand. Under a specific set of binding and washing conditions some beads, usually a very small fraction, bind a high amount of the target, some beads bind an intermediate amount of the target, and some beads bind a low amount of the target. The vast majority of beads bind essentially no, or background levels of the target, indicating little or no specific binding. For each high affinity ligand present in a library there may exist many analogs within the library that might still bind to the target, but with lower affinity and/or specificity. Nevertheless, for each individual target, the amount that binds to the library is a function of its relative concentration in one. sample versus another, so that more target is bound from a sample with a higher initial concentration of that target than is bound from a second, otherwise comparable sample, which has a lower initial concentration of that target.

At very low target concentration, only ligands with very high affinity and specificity will bind the target. The result of increasing the amount of analyte over a broad range of concentrations is that it will bind to an increasing number of additional and possibly lower affinity ligands and will compete more effectively with proteins that compete for binding to ligands with affinity for multiple proteins. Thus, given sufficient diversity in the library and complexity of the starting material, there will be increased binding of the target over a very broad concentration range, in theory only leveling off when all the beads that bind a given target are saturated and the target has competed with all other targets present for binding to ligands with common affinity.

To enrich further for the binding of trace targets, additional affinity ligands specific to highly interactive proteins that preferentially bind to a large number of beads within a library, (e.g. fibrinogen, albumin, immunoglobulin, HDL, LDL, etc.), may be included in the contact step to sequester the most interactive proteins away from the library. For example, the specific beads may be included in a compartment separated from the library by a dialysis membrane freely permeable to proteins, but impermeable to the beads. The specific affinity ligands will then preferentially sequester a high percentage of the promiscuously-binding proteins, thereby allowing trace proteins, whose binding to library ligands may be impeded by the most interactive binders, access to other library ligands. The advantages of performing this partitioning by methods such as equilibrium affinity dialysis instead of conventional depletion strategies is that targets binding to the major species can still be captured on the library without being lost along with the depleted highly interactive species. In addition, a proportion of the most interactive proteins will remain in solution to bind to high affinity binding sites within the library and will still be analyzed, albeit at a lower overall concentration. Several high affinity resins specific for different proteins may be included in the same compartment.

The resins for the most interactive species may be used to sequester these species in additional formats to equilibrium dialysis described above. For example, the beads bearing ligands that bind to the most interactive species may be magnetic and may be separated based on magnetic charge. Alternatively, the beads may be physically separated by sedimentation rate, density, and size. Alternatively, the beads or ligands may be fixed to supports such as dip-sticks, and membranes. The amount of the target-specific ligands required may be deduced by knowing the amount of target to be removed, its affinity for the resin and the equilibrium concentration.

Thus, the invention teaches that protein concentrations may be analyzed over a very broad range. Moreover, the degree of competition for binding may be modulated by reduction in the free concentration of the most interactive targets by equilibrium dialysis using high affinity ligands to these targets.

Any one analyte may be concentrated by affinity chromatography using a ligand that has, for all practical purposes, selective and high affinity for essentially only that one analyte. In this regard, monoclonal antibodies have been extensively used and are frequently immobilized on a support, e.g. microtiter plate or an array. The analyte may be captured until it saturates the binding sites. The amount of analyte bound to the ligand increases with increasing concentrations of analyte in the starting material. This can be represented by the formula:
Specific binding=B=Bmax[analyte]/([analyte]+K_{D})
where B is the amount of bound analyte and Bmax is the maximum amount of analyte that can be bound to the available ligands.

The amount of analyte bound is also dependent on the sample volume, V, and the relative mass of the affinity particles to which they bind, M. Such that:
[analyte]V+MB=[analyte initial]V
In practice the frequency of detecting beads within a library that bind an individual target with high affinity, in our experience, varies from about 1:5,000 to 1:>1,000,000 and for most practical purposes V is significantly greater than the total volume of library. Hence, V is >>M and [analyte]V is >MB. Thus, the amount of analyte in solution usually does not decrease significantly during the capture process.

This formula for binding produces a sigmoidal dose response curve of the amount bound as the ordinate is plotted against analyte as the abscissa, i.e. a binding isotherm with a plateau at Bmax. The K_{D} is the equilibrium dissociation constant. The total amount of analyte bound will depend on the number of analyte-binding beads and their capacity as well as the initial concentration of analyte in the sample, the initial volume of the sample, and the capacity of the analyte-binding beads. In this simple model about 90% of the equilibrated concentration range for binding will be covered in an 81-fold range in the analyte concentration assuming that the concentration of the free analyte after equilibrium binding is approximately the starting concentration of analyte. Beyond this range, large changes in analyte concentration would be expected to have little impact on the amount of bound analyte. In contrast, a mixture of affinity resins with differing equilibrium dissociation constants for an analyte spanning many orders of magnitude will provide a broad increase in the amount of analyte bound over very many orders of magnitude of analyte concentration, i.e.
B={(Bmax1[analyte])/([analyte]+K_{D1} )₁+(Bmax₂[analyte])/([analyte]+K_{D2} )₂. . . n}+NS
where 1 and 2 are two of n, number of different beads with varying affinities (K_{D1} 1, K_{D2} ) for the analyte and NS is non-specific binding of the analyte. In general, as the surface area of different beads within our library is usually similar for a given analyte, the total capacity, Bmax₁, may be similar to Bmax₂.

A library of sufficient complexity would be expected to contain a large number of individual affinity beads covering a range of dissociation constants that span very many orders of magnitude. Thus, the amount of analyte bound at different concentrations of free analyte, in the absence of competing entities, will be given by the sum of a large number of different sigmoidal curves resulting in a very broad dose response curve.

However, within a library of sufficient complexity there are ligands that bind to multiple targets such that they compete with each other for binding according to their relative affinities for the different targets to which they bind and the concentrations of the targets according to the laws of mass action. Thus, in a simplified system, an “analyte1” will compete with a reversible inhibitor, “analyte2” according to the equation:
n₁=n₁max.K₁ₐC₁/(1+K₁ₐC₁+K₂ₐC₂) n₂=n₂max.K₂ₐC₂/(1+K₁ₐC₁+K₂ₐC₂)

and the mass balances are
VC₁i=VC₁+Mn₁VC₂i=VC₂+Mn₂
Definitions
Initial concentration of analytes 1 and 2: C₁i and C₂i
Concentration of 1 and 2 in equilibrium with resin: C₁i and C₂
Mass of 1 and 2 bound per mass of resin: n₁ and n₂
Volume of sample and mass of resin: V, M
Association constants for 1 and 2: Kᵢₐ and K₂ₐ
Capacities for 1 and 2: n₁max and n₂max

Thus, the mass of n₁bound will decrease as the term K₂ₐC₂ increases. The latter is dependent on both the association constant Ka and the concentration of the competing species c₂ Thus, the ability of analyte2 to compete with the binding of analytel will be more effective as the concentration of 2 increases over a very broad range and not just for the lowest concentrations.

The net result of increasing an analyte concentration over a broad range is that it will bind to an increasing number of lower affinity ligands and will compete more effectively with other proteins that compete for binding to the same ligands. Thus, given sufficient diversity in the library and complexity of the starting material there will be increased binding of an analyte as its concentration range increases, in theory only leveling off when all the beads that bind a given analyte are saturated and the analyte has competed with other proteins for binding to ligands in common.

As mentioned above, a complete modeling of the amount of a trace component in a complex mixture that will bind to a combinatorial library of ligands has not been undertaken and must take numerous other considerations into account in addition to relative analyte concentration and ligand affinities and specificities.

Reduction of most-interactive proteins

In the example of binding a target to an affinity library, the sample volume is usually extremely large compared to the volume of the actual volume of beads that bind to a specific target, leaving the free concentration of target relatively unaffected by binding of the target to the library. Decreasing the concentration of the most interactive targets bound to the library may allow more rarely-binding proteins to access ligands. This can be accomplished by decreasing the overall free concentration of these species through adding sufficiently high amounts of selected affinity supports which are highly specific to, and have high affinity for, a highly interactive target to capture the majority of the target in the starting material. Such that MB for this species is >>[analyte]V. Under these conditions, the free analyte concentration is dramatically decreased which in turn decreases the amount of the most interactive targets bound to the library according to the equation:
Specific binding =B=Bmax[analyte]/([analyte +K_{D} )

Thus, to remove the most interactive target(s) with high binding potential to the library of ligands it is necessary to separate physically the support(s) that bind the most interactive target(s) from the library. This may be achieved by means of separating the supports in 2 interconnected compartments (one containing the combinatorial library, one containing the support(s) that bind the most interactive analytes), such as a single compartment with a semi-permeable barrier (large porosity dialysis membrane), or using specific ligands coupled to magnetic resins, dipsticks, etc. Multiple selected ligand-support complexes to a number of species with the most interactive binding potential may be used simultaneously in combination with each other.

Ligands

For purposes of the invention, the term “ligand” as used herein refers to any biological, chemical, or biochemical entity, such as a compound that binds to a target. It is important to note that two or more targets can compete for binding to one or more ligands. The ligand can be provided by isolation from natural or synthetically produced materials. Suitable ligands for the inventive method include, but are not limited to, amino acids, peptides, nucleic acids, antibody preparations (e.g. antibody fragments, chemically modified antibodies, and the like), carbohydrates, sugars, lipids, steroids, drugs, vitamins, cofactors, organic molecules, and combinations thereof.

Organic molecules include, for example, synthetic organic compounds typically employed as pharmacotherapeutic agents. Such molecules are, optionally, mass produced by combinatorial methods or, by strategic syntheses devised to arrive at specific molecules. Likewise, organic molecules also include natural products and analogues, whether extracted from their natural environment or strategically synthesized. The term “organic” as used herein is not intended to be limited to molecules comprised only of carbon and hydrogen, but rather is used in its broader sense encompassing macromolecules of biological origin.

Preferably the ligands are peptides. More preferably, the peptides consist essentially of about 2 - 15 amino acids. The term peptide as used herein refers to an entity comprising at least one peptide bond, and can comprise D and/or L amino acids. Ideally the ligand is between 3 and 10 amino acids. If desired the peptide can be generated by techniques commonly employed in the generation of combinatorial libraries, e.g. the split, couple, recombine method or other approaches known in the art (Furka et al, Int. J Peptide Protein Res., 37:487-493 (1991); K. S. Lam et al., Nature, 354:82-84 (1991); WO 92/00091 (1992); U.S. Pat. No. 5,133,866; U.S. Pat. No. 5,010,175; U.S. Pat. No. 5,498, 538). Expression of peptide libraries is described by Devlin et al., Science, 249:404-406 (1990). Combinatorial libraries are libraries of diverse peptides (sometimes of a specific length, sometimes of various lengths). In peptide libraries, the number of discrete peptides of different sequences increases dramatically with the number of cycles of coupling reactions performed and the number of separate reactions per cycle. For example the random incorporation of 19 amino acids into pentapeptides produces up to 2,476,099 (195) individual peptides of differing sequence (Lam, K. S., et al., Nature 354:82-84 (1991)). Combinatorial methods allow generation of combinatorial libraries of ligands directly on a support. Typically the ligands are synthesized on particles of support media such that multiple copies of a single ligand are synthesized on each particle (e.g. bead), although this is not required in the context of the invention. The preferred density of the ligands is 50-400μmol/gram dry weight, more preferably 50 to 150 lmole/gram dry weight, and most preferably 100 ttmole/gram dry weight.

Examples of amino acids that may be included in the library are the naturally occurring L-amino acids and their D-enantiomers. Other amino acids include unnatural amino acids such as: 2 or 3-aminodipic acid, beta-alanine, 2-aminobutyric acid, 6-amino caproic acid, citrulline, hydroxylysine, N-methylvaline, and norleucine. The amino acids may be modified, for example, through phosphorylation of serine, threonine and tyrosine. In addition, the ligand library may be modified post-synthesis by chemical or biochemical means. Examples of chemical modifications include acetylation of amino groups with acetic anhydride, reaction with aziridines, epoxides, and methylglyoxal. Some modifications are the result of Maillard reactions and such products in tissue proteins are implicated in the pathology in aging, e.g. advanced glycation end-products and glyoxidation products such as N⁸-(carboxyethyl)lysine. Other modifications may be enzymatic through the action of protein kinases that phosphorylate serine, threonine and tyrosine, and glycosylases that glycosylate asparagine, serine and threonine.

Ligand-support complexes specific to abundant species may be identified by screening combinatorial libraries as described in U.S. patent application Ser. No. 10/414,523 and U.S. patent application Ser. No. 10/601,032. Alternatively, they may be obtained from other methods including hybridoma technology and from commercial sources. Such commercial suppliers include Agilent, Genway and Sigma.

Target

For purposes of the present invention, the term “target” and “analyte” as used interchangeably herein refers to any chemical, biochemical or biological entity, such as a molecule, compound, protein, virus, microparticle, organelle or cell, that binds to a ligand. The target can be isolated from nature or synthetically produced, and can be organic or inorganic in nature (e.g., a synthetic inorganic compound or a synthetic organic compound). For example, the target can be a drug or drug candidate (such as a small molecule drug candidate), a toxin, a fertilizer component, an insecticide, or a derivative, analogue or enantiomer thereof. In addition, the target can be endogenous or exogenous to any prokaryote or eukaryote, e.g. bacterium, a fungus, yeast, a plant, or a mammal. Suitable targets for the inventive method include, but are not limited to, cells (be they eukaryotic (such as mammalian cells, e.g. stem cells or cells in culture, yeast cells and plant cells) or prokaryotic (such as bacteria cells and archaea cells), viruses, microparticles, organelles, proteins, protein complexes, peptides, amino acids, nucleic acids, isoforms of any of the foregoing, and combinations of any of the foregoing. By isoforms it is intended to mean proteins, protein complexes, peptides, and nucleic acids that differ from the native protein, protein complex, peptide or nucleic acid. Such a difference can be structural, in which the primary amino acid sequence is the same, but the three-dimensional structure differs. Preferably, the targets are proteins. Suitable proteins targets include, for example, receptors, antibodies, immunogens, enzymes (e.g. proteases), growth factors, cytokines and enzyme substrates. More preferably, the proteins are found in blood, saliva or urine, either as endogenous components, or as the product of cellular breakdown, e.g. microparticles and other biomarkers of disease, and infectious agents such as prion. Such proteins in plasma include, for example, normal prion protein, proteases, epitope-specific antibodies, complement factors, fibrinogen, API, or coagulation factors, all of which are naturally found in the blood of an organism in a non-diseased state. Alternatively, the blood protein is present in plasma associated with a diseased state (optionally not found in the plasma of a healthy subject) or as a result of the administration of an agent, e.g. a drug. Alternatively, a target may be found at higher or lower concentrations in a diseased state compared to a normal state, or may be modified or post-translationally altered in the disease state. In this regard, the plasma protein can be a PrPsc prion protein associated with a transmissible spongiform encephalopathy.

The target for the inventive method can be obtained from any source. A sample comprising the target can be a complex solution, such as an environmental sample or extract selected from soil, air, water (naturally occurring or man-made), food, and swabs for evaluating environmental contamination (for example, swabs from a building), and the like. The sample comprising the target can be a composition comprising chemical compounds or synthetic mixtures of compounds and can be present in a combinatorial library and/or present in organic solvents under extreme conditions of pressure, temperature, etc. Preferably the targets are present in, or isolated from, a biological fluid. By “biological fluid” is meant any aqueous solution obtained directly from a prokaryote or eukaryote (i.e. whole blood, plasma, pooled plasma, intermediates from pooled plasma, biological products, serum, a cell homogenate, a conditioned medium, a fermentation broth, cerebrospinal fluid, urine, saliva, milk, ductal fluid, tears, perspiration, lymph, semen, etc.). The biological fluid can be obtained directly from a prokaryote or eukaryote, such as blood, sweat, tears, lymph, saliva, and urine. Alternatively, the biological fluid can be obtained from culturing cells of the organism, such as a fermentation broth and cell culture medium. Suitable biological fluids for use in the inventive method include, but are not limited to, blood, plasma, serum, a cell homogenate, a tissue homogenate, a conditioned medium (a cell culture medium (lacking cells) that is collected after it has been incubated with the cells), a membrane preparation, inclusion bodies, a platelet preparation, a fermentation broth, cerebrospinal fluid, urine, saliva, milk, ductal fluid, tears, perspiration, lymph, exudates from wounds, and semen. Preferably, the biological fluid is blood, or plasma. The biological fluid can be obtained from a host afflicted with a disease, or from a healthy control. The targets may be liberated into the biological fluid as a result of tissue damage.

One advantage of the inventive method is the ability to identify and/or characterize and quantify targets on the basis of chemical, biochemical and biological activity, without prior knowledge of the target's molecular identity. The chemical activity may be a mass spectral signal and the biochemical activity may be an enzyme activity such as a protease, organophosphatase, an inflammatory cytokine, etc. Biological activities include anti-infective activity such as bactericidal and antiparasitic activity and the growth, death, and differentiation of cells as well as similar and additional effects on eukaryotic organisms.

In a preferred embodiment the test sample is whole blood and the targets plasma proteins. Whole blood requires the presence of anticoagulants to prevent coagulation over time. Preferred anticoagulants include EDTA, citrate, heparin and protease inhibitors such as aprotinin, and D-phenylalanyl-L-propyl-L-arginine chloromethylketone (PPACK), etc. The preferred contact time is kept as short as possible to prevent undue protein modification over time. A contact time of <15 min is possible with other times ranging up to and beyond 24 hours. The preferred ratio of combinatorial library to whole blood may be in the order of 1:1 to 1:I0 to 1: 1,000 or more.

A further advantage of the targets being immobilized as target-ligand-support complexes is that when ligands bind to targets, i.e. proteins, the targets'thermal stability is increased in many cases (see U.S. Pat. No. 5,786,458 to Baumbach, Hammond, Lang and Galloway, for examples and references). Although this was exploited for improved and specific viral inactivation of therapeutic proteins, the same general principles will be true for the multiple targets bound to multiple ligands in this invention. Thus, the inventive method will simultaneously bind and concentrate trace plasma proteins from blood without the need for generation of plasma through centrifugation or serum collection by clot formation. Furthermore, it will decrease the interference caused by the abundant species in blood including whole cells and abundant proteins such as albumin and transferrin, while immobilizing the analytes on the ligands. Following removal of the non-bound proteins immobilization of the proteins will physically restrict their ability to diffuse freely into solution to interact and potentially for the proteases to degrade important biomarkers. In addition, immobilization of proteins may stabilize their structure further preserving the ability to evaluate chemical, biochemical and biological properties.

Supports

In one embodiment of the inventive method, the ligand is attached to a support. The term “support” as used herein refers to any support matrix, such as those solid supports known in the art, which serve to immobilize the ligand. Suitable supports include, but are not limited to, membranes, filters, meshes, beads or particles comprised of or coated with cellulose, acrylates, polyacrylates, polyhydroxymethacrylates, polystyrene, dextran, agarose, polysaccharides, hydrophilic vinyl polymers, polymerized derivatives of any of the foregoing and combinations of any of the foregoing, as well as any porous or non-porous matrix on which ligands can be synthesized or immobilized. By “derivatives” it is meant a substance related structurally to another substance and theoretically derivable from it. Preferably, the support is inert such that any possible chemical reaction with the target, target-containing starting material, and/or ligand is minimized. Preferably the support is biochemically inert such that fumctional proteins, e.g. complement and coagulation proteins in blood, are not activated by the support. Preferably the support is biologically inert such that cellular function is unaffected by the support. Most preferably the support can be used directly with whole blood without the need for prior fractionation to remove red and white cells, platelets, lipids, microparticulates, and antibodies.

The criteria of selection of a base polymer for use as the support for synthesizing the combinatorial library is that it should be chemically, biochemically and biologically inert. Preferred supports are resin beads comprising a material selected from the group consisting of agarose, cellulose, dextran, ethylene glycol, fluoropolymers, polyacrylate, polyesters, polyethylene glycol, methacrylate and hydroxymethacrylates including glycidol methacrylate, ethylene glycol dimethacrylate, pentaerytritol dimethacrylate, dimethacrylate, and methacrylate monomer, polypropylene, polyethylene oxides, polysaccharide derivatives of any of the foregoing, and combinations of the foregoing. A particularly preferred support material is a polyhydroxylated methacrylate polymer. Examples of such resins include Toyopearl AF-Amino 650M from Tosoh Bioscience, fractogel EMD Amino (M) from MerckKGaA in Darmstadt, Germany, and Affi-Prep and MacroPrep media from Bio-Rad. The resin should also possess sufficient concentration of fimctionalized groups for the chemical synthesis of combinatorial libraries by the split, couple and recombine method of Furka et al. as extended by Lam et al. Furka et al., Int. J Peptide Protein Res. 37:487-493 (1991); Lam et al., Nature 354:82-84 (1991).

Many solid supports displaying potential ligands are commercially available. Alternatively, the one or more ligands of the inventive method can be indirectly attached or directly immobilized on the support using standard methods (Merrifield, R. B. J. American Chemical Society 85(14):2149-2154 (1963); Harlow and Lane, Antibodies, Cold Spring Harbor Laboratory, Cold Spring Harbor, NY (1988); Biancala et al., Letters in Peptide Science, 7(291), 297(2000): MacBeath et al., Science, 289, 1760-1763 (2000); Cass et al., ed., Proceedings of the Thirteenth American Peptide Symposium. Leiden, Escom, 975-979 (1994); U.S. Pat. No. 5,576,220; Cook et al., Tetrahedron Letters; 35, 6777-6780 (1994); and Fodor et al., Science, 251(4995): 767-773 (1991)). In one embodiment, the ligands are provided by synthesizing them on the surface of a support, which is advantageous in generating peptide libraries. Alternatively, the ligands can be provided by chemically conjugating them to the support or can be attached via linkers, such as streptavidin, beta-alanine, glycine, methionine, polymers containing glycine and serine, (—O—CH₂—CH₂—)n where n is a number between 1 and 30, short chain hydrocarbons of the formula —CH₂)—, polyethylene glycol, and epsilon amino caproic acid. The linkers may also comprise (0—13 CH₂—CH₂)n where n is 1 -30.

In the context of the inventive method, at least a portion of the targets of the target-ligand-support-complexes are usually dissociated from the ligand-support-complex. By eluting or dissociating at least a portion of the targets, it is meant that a percentage (or fragment) of any one specific target is eluted, since it is unlikely that 100% of the target bound to a specific bead could be transferred. Thus, by “at least a portion” it is meant that at least 10%, 20%, 30%, 40%, 50%, 60%, 70%, 80% or 90%, of the target of at least one target-ligand complex.

The dissociation is achieved through contacting the target-ligand-support complexes with a solution that promotes dissociation. The solution can be selected from buffers of known salt concentrations (2M NaCl), extremes of pH, or denaturing capability, e.g. strong chaotropes (6M guanidine HCl), organic solvents, de-ionized water. Alternatively, or in addition, an isoelectric gradient can dissociate the target from the ligand-support complex. Transfer solutions can also comprise ligands (different from the ligands on the ligand-support complexes), cofactors for the target, enantiomeric specific molecules, and the like. Use of different transfer solutions allow investigation of elution conditions and targeting sub-populations of target-ligand interactions.

The dissociation conditions employed in the inventive method are selected to minimize disruption of the ligand from the support. In other words, the elution and transfer conditions should not release the ligand (or ligand-support complex) from the matrix (unless this is specifically desired).

Contact conditions

The sample containing targets may be mixed with the ligand-support complexes in one of several different formats as taught in the parent applications (U.S. patent application Ser. Nos. 10/414,523 and 10/601,032). These formats include chromatography column formats, batch addition of ligand-support complexes, monolithic structures, membranes or arrays. The beads may be macroporous and may be milled to a fine powder.

Binding of the trace components may be improved by binding to a library in one compartment while ligands that exhibit high affinity and capacity to the most interactive bound targets may be separated in another compartment. Such an embodiment can be achieved by placing the support in two interconnected chambers (one chamber having ligand-support complexes that bind the most interactive targets, one chamber having a combinatorial ligand-support complex library), use of equilibrium dialysis or the use of magnetic beads or dipsticks coated with a ligand. Equilibrium dialysis equipment is commercially available from companies such as Harvard Apparatus and SDR Molecular and may possess 2 or more chambers. Magnetic beads may be made by incorporating micronized magnetic particles into the synthesis of the resin or modifying the beads through reaction with magnetic particles. Such beads are available from BioScience Bead Division of CSS International. Magnetic beads may be easily separated from non-magnetic beads. Likewise, the specific affinity ligands can be included on the surface of dip-sticks or may be synthesized on, or coupled to the surface of a membrane. Physical separation may then be easily accomplished by physically removing the dipstick or membrane with the target-ligand-support complexes attached.

Preferably the capacity of the ligand support complexes to the most interactive analytes, should be in excess capacity (>10 fold) over that of the analyte to be bound. More preferably it could be >100 fold. The ligand ideally should have a relatively fast association rate and may be contacted with the test sample in advance of the contact with the library. The temperature and binding conditions should preserve the conformational structure of the analytes in the sample. For proteins, the temperature would be typically between 0° C. and 60° C. and more preferably between 4 ° C. and 40° C. It may be at physiological temperature, e.g. about 37° C. from mammalian species. The pH and isotonic conditions are also preferably selected to maintain protein structure and function with a pH range of 3.5 to 10 and more preferably 6-8. For human blood a pH of about 7.4 may be used. The test sample may be prepared in a salt solution which preserves protein structure, a range of 0.05 to 2 M NaC₁i or equivalent may be used while a physiological concentration of 0.15 M NaC₁i is preferred for human blood. The solution may be buffered with phosphate, HEPES, imidazole, histidine, Tris, Triethanolamine, acetate, citrate and other buffers. For blood and blood derivatives an anticoagulant is preferably included. Such anticoagulants include citrate, EDTA, and heparin plus PPACK, or a cocktail of protease inhibitors. A contact time of <15 min. is possible with other times ranging up to and beyond 24 hours. The sample may be precontacted with the selected resins targeting the most interactive proteins.

The preferred ratio of volume of combinatorial library to volume of whole blood may be in the order of 1:1 to 1:10 to 1:100 to 1: 1,000 or more.

Detection and analysis of the target

The inventive method further comprises detecting the dissociated targets that bind to the ligands of the ligand-support complexes. The term “detection” and words related thereto as used herein refer to the identification of any distinctive quality or trait of a target, and do not require that the precise chemical identities (e.g. the molecular formula, chemical structure, nucleotide sequence or amino acid sequence of the target) is elucidated. Furthermore, detection of multiple targets may be performed individually, sequentially or simultaneously. Alternatively, the targets can be detected by testing for a property or activity of the target, such as a biological property, chemical property, or a property that is a combination of any of the foregoing. The targets may be directly detected using, for example molecular weight by mass spectrometry or gel-electrophoresis, or spectral signal. Alternatively, the targets may be detected using immunological assays, for example, ELISA, Western blot and nephelometry assays. Otherwise, the targets may be detected by means of an enzyme assay such as a protease or organophosphatase that hydrolyses a fluorogenic substrate to create a fluorescent signal. On the other hand, the targets may be detected and analyzed by contacting cells with the eluted targets and detecting a cellular response using a biological assay such as cell growth, death, and differentiation. Additional techniques for detection and analysis are reviewed in Phizicky EM and Fields S. 1995, Protein-Protein Interactions: Methods for detection and Analysis, Microbiological Reviews, 59, (1) 94-123. See also the parent applications (U.S. patent application Ser. Nos. 10/414,523 and 10/601,032).

Pathogens including viruses may be bound to the ligands of combinatorial libraries (U.S. patent application Ser. No. 10/601,032) and detected by plaque formation around a bead binding the virus. The virus may alternatively be detected following binding to beads by lysing the virus and transferring the liberated viral DNA onto a membrane, where it is detected by labeled cDNA probes complementary to the viral DNA. Probes may be radiolabelled or biotinylated. In the latter case, binding of the probe is visualized with streptavidin-alkaline phosphatase. We have demonstrated this for parvoviruses (data not shown). Alternatively the viruses can be eluted from the combinatorial library en masse and detected by means of either its infectivity in an appropriate assay, e.g. based on plaque formation in a susceptible cell line or by a nucleic acid amplification technique such as polymerase chain reaction. Using this method the virus can be concentrated, and substances that can interfere with PCR removed by washing thereby making diagnostic assays for viruses from complex samples such as whole blood, more sensitive. The product of the reaction may be analyzed by sequencing of the amplified nucleic acid to confirm the identity and isotype of the virus.

The present invention also provides a method of identifying a diagnostic marker. In a preferred embodiment, the method comprises the steps of (a) providing a first set of samples from a first set of organisms having a first phenotype, (b) providing a second set of samples from a diseased individual having a second phenotype, (c) performing the method for reducing the range in concentrations and preserving the relative amount of analytes initially contained in each of the samples, thereby creating a third and fourth set of samples, respectively; (d) detecting analyte species in each of the third and fourth set of samples, whereby the at least one analyte species and its approximate concentration is a biomarker for distinguishing the first phenotype from the second phenotype.

### EXAMPLES

### Example 1

Fibrinogen and API are both abundant plasma proteins, present at about 1-2 mg/ml; however, previous work in identifying ligands for these proteins has demonstrated that the number of high affinity ligands present in a typical library for each varies by orders of magnitude. High affinity and high capacity fibrinogen ligands have been estimated to be present at about 1:5,000 ligands, whereas high affinity and high capacity API ligands may be present at only 1: 1,000,000 ligands. The purpose of this experiment was to examine the relative ratio of fibrinogen and API in untreated and treated plasma, to see if the difference in ligand number affects the amount of each that is recovered from the library.

Methods

Frozen platelet-poor plasma (PPP) was thawed at 37° C. and filtered through 0.8 μm and 0.45 μm filters. Approximately 1 ml Toyopearl 650M library (Toyopearl AF-Amino 650M resin with peptide ligands of varying lengths synthesized thereon) was incubated with 9 ml filtered PPP pool for 1 hour/RT/rotating. The resin was washed and the first 1 ml of wash buffer (Citrate buffer: 20 mM citrate, 140 mM NaC₁i, pH 7.4), as well as the flow through were collected for analysis. An aliquot of the resin volume (˜220μl) was heated with 1/1 (v/v) 2x (LDS buffer+DTT reducing agent) for 10 minutes at 90° C. The supernatant was collected and saved for analysis and called “LDS buffer eluate”.

A 1:500 dilution of the initial PPP was heated in LDS buffer+dithiothreitol (DTT) reducing agent for 10 minutes and 20 jil of this sample and 20 μl of a 1: 40 dilution of the eluted sample was loaded on a 4-12% Bis-Tris gel. A 1:50 dilution of the initial PPP along with a 1:4 dilution of the eluted sample was also loaded in separate set of lanes on the same gel. The gel was electrophoresed in MOPS buffer at 200 V, until the dye front reached the bottom of the gel.

Protein was transferred from the gel onto a PVDF membrane for 45 minutes at 100V in NuPage transfer buffer (Invitrogen). Non-specific binding of proteins to the membrane was blocked for 1 hour in Western Breeze reagents (Invitrogen). The membrane was cut in half. One half was incubated in Mouse Anti-Fibrinogen (gamma chain) antibody (cross-reacts with human) at 1:5,000 in Western Breeze reagents over night at 4° C. The other half was incubated in mouse anti-human API at 1: 10,000 in Western breeze reagents over night at 4° C. Each half was washed for 3x 10 minutes in Western Breeze wash and incubated with 1:10,000 goat anti-mouse-alkaline phosphatase in WB reagents for 1 hour. The membranes were developed with CDP Star, and exposed to X-Ray film.

Results:

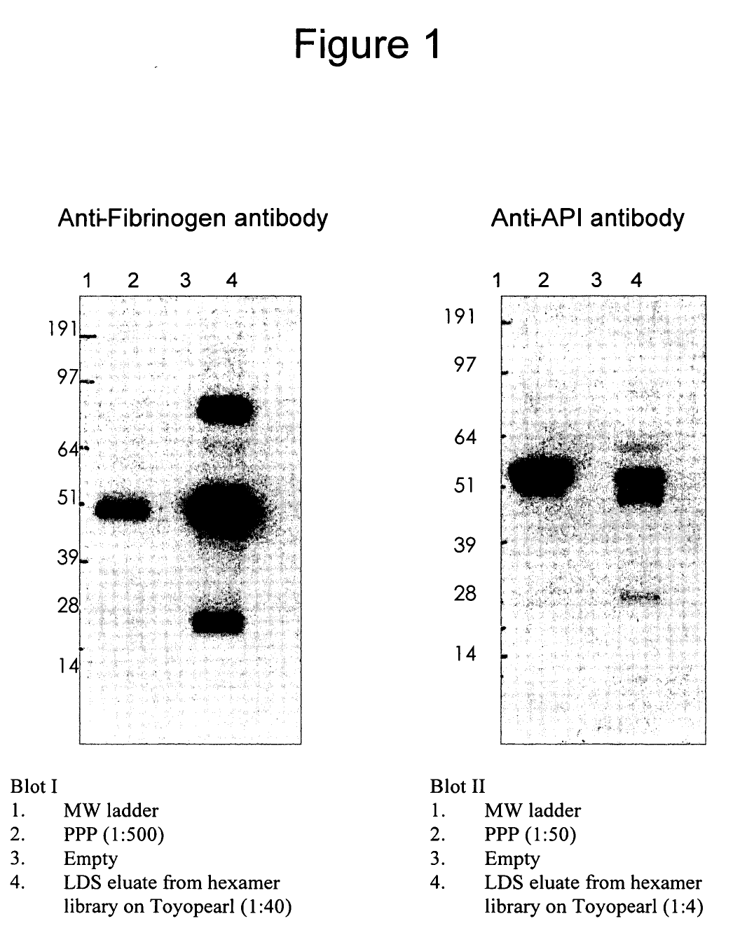

In lanes 2 of both gels (see FIG. 1), correcting for the 10x dilution difference, the signal from the starting fibrinogen and API are very broadly consistent with their known concentrations in human plasma. Following binding to the library substantially less API is recovered from the beads and detected in the Western blot compared to the starting material. Specifically, the signal from API (anti-API, lane 4), even with 12x more eluate loaded on the gel (1:4 versus 1:50), is weaker than the starting plasma. Additional bands, perhaps representing trace components that were not visible in the starting material, appear in lane 4 consistent with our previous observations that trace analytes are enriched on the ligands (see U.S. patent application Ser. No. 10/414,523 and U.S. patent application Ser. No. 10/601,032). Thus, API itself is dramatically under-represented on the resin compared to the starting plasma. Fibrinogen gamma chain, which represents about one third of the total fibrinogen, whose original concentration in plasma is equivalent to API, is relatively weak in plasma. The relative signals of API and fibrinogen from plasma can not be directly compared as they used different primary antibodies with different titers and affinity; however, the concentration of fibrinogen gamma chain in the eluate from the library is dramatically stronger than the starting material even when considering the relative dilution. This contrasts dramatically to the situation for API were the converse is seen. For these two proteins, and others that result in the same phenomenon, the concentration range of proteins in the eluted fractions is increased, not decreased. These results also demonstrate that the number of high affinity ligands that are present in a library depends on the specific characteristics of the protein, not solely its abundance.

### Example 2:

Differential concentration of virus based on original titer

The amount of binding of any one analyte to a library of ligands is a function of several factors including the concentration of that particular analyte in the sample. Samples with a higher original concentration of the analyte purify more of the analyte than samples with a lower original concentration of the analyte. These experiments examined if this phenomenon was also true for binding virus.

Methods

10 ml of plasma or phosphate buffered saline (PBS) was spiked with porcine parvovirus (PPV) or human parvovirus B19 virus at the indicated titers (see Table 1). Spiked samples were incubated with 100 μl of the Toyopearl 650M library (see Example 1) for one—1.5 hours at room temperature. Bound virus was eluted from the resin by adding 1 ml 30 mM sodium citrate pH 3.0, mixing, centrifuging the resin and collecting the supernatant. The total amount of virus eluted from the resin was quantified either by PCR to measure genome copy equivalents (GCE) or by infectivity assay to determine the (50% Infectious DoseTClD₅0).

TABLE 1

| | | | |
|---|---|---|---|
| | Starting | | |
| | Titer | Eluted Titer | |
| Virus | (log/ml) | (log) | Assay |

| | | | |
|---|---|---|---|
| PPV (plasma) | 5.85 | 5.95 | GCE |
| | 5.69 | 4.58 | GCE |
| | 3.75 | 4.03 | TCID₅₀ |
| | 2.62 | 2.42 | TCID₅₀ |
| | 1.83 | 1.91 | TCID₅₀ |
| | 1.74 | 1.81 | TCID₅₀ |
| B19 (buffer) | 7.53 | 8.46 | GCE |
| | 5.66 | 6.06 | GCE |

Results

Table 1 is a compilation of several experiments in which plasma or buffer containing different titers of either PPV or B19 were incubated with the resin. For both viruses, the amount of virus eluted from the resin, is proportional to the concentration of the original sample, reported per ml. The presence of plasma proteins did not interfere with this proportional enrichment.

The average starting volume of plasma is about 10 times the resin volume. On average the amount of protein in plasma is over 60 mg/ml and that bound to the resin is about 10 mg and that eluted at pH 3 is about 3 mg. Consequently, the actual amount of virus bound and eluted from the resin is in about 3/600 or 200 fold less total protein which represents a significant increase in specific activity over the starting levels.

### Example 3

### Use of combinatorial libraries for improved troponin detection by Western Blotting

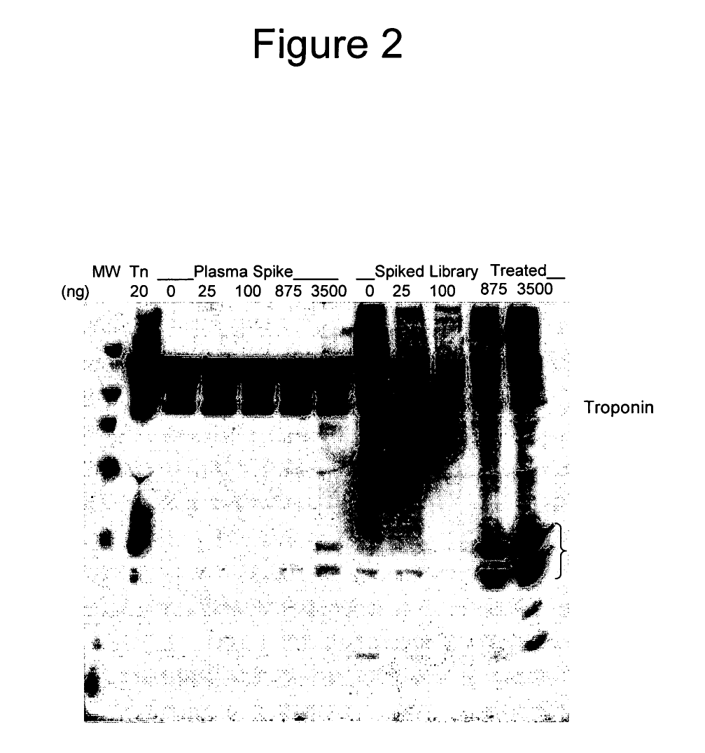

Troponin (Biodesign, Cat #A86862H Lot # 3C07903) was spiked into an aliquot of human whole blood at the following concentration: 0, 25, 100, 874 and 3,500 ng/ml. Blood was fractionated by centrifugation and the plasma collected from one part of the blood spiked samples (Plasma Spike). The second part of blood samples (Spiked Treated) was incubated with 100 JAl of the Toyopearl 650M library (see Example 1). The Toyopearl 650M library was swollen in DMF, washed with 20% methanol (MeOH) and stored in 20% MeOH. Immediately before the experiment the Toyopearl 650M library was washed with PBS and equilibrated with citrate buffer. After incubation with blood samples, the library was washed three times with 1 ml of PBS to remove non-bound proteins. Bound proteins were eluted with 255μl of 0.05M HC₁i and immediately neutralized with 85 ~l 0.5M NaH₂PO₄ pH 7.5. Protein concentrations were evaluated in all samples, equal amount of protein (33 μg) of eluates obtained from library, i.e. spiked blood and plasma samples were loaded per lane of the gel for Western Blot analysis. Western Blot was performed according to standard procedure. Proteins were transferred onto PVDF membranes, the membranes blocked for non-specific binding and then stained with primary anti-Troponin I antibody (Biodesing, cat #H86207). Following incubation with primary antibody and washing, secondary goat anti-mouse IgG antibody labeled with peroxidase was added, further incubated and washed to remove non-bound antibody. WesternBreezeTM Chemiluminescent Detection Kit (Invitrogen) was used for chemiluminescent detection of peroxidase. The results are shown in FIG. 2 and demonstrate that library-treatment significantly improves troponin detection in plasma samples. Moreover, the differential in troponin concentration between the different samples is maintained during library binding and elution.

### Example 4.

### Use of combinatorial library for improved troponin detection and analysis by ELISA

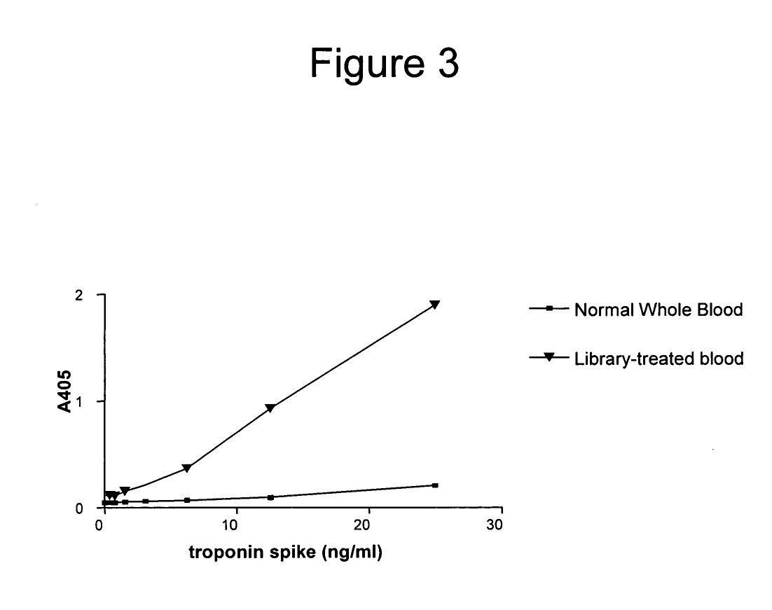

Troponin (Biodesign, Cat #A86862H Lot # 3C07903) was spiked into human plasma or human citrated blood at different concentrations (0-1OOng/ml). Toyopearl AF Amino 650 M library (see Example 1) was swollen in DMF, washed with, and stored in 20% MeOH. The library was washed with PBS and equilibrated with citrate buffer directly before the experiments were performed. Blood samples (1 ml) were added to the column containing resins (100 μl of bed volume) The resin was washed three times with 1 ml of PBS to remove non-bound proteins. To remove extra PBS, columns were centrifuged for 4000g for 1 min in an Eppendorf tube. Bound proteins were eluted with 250 pl of 0.05M HCl and immediately neutralized with 85 μl 0.5M NaH₂PO₄ pH 7.5. Fifty ill of 1% BSA with 0.05% Tween 20 was added to stabilize the proteins. Evaluation of troponin in plasma and library treated blood samples was performed by Troponin I ELISA, Bio-Quant kit (#BQ 015C) according to the manufacturer's manual. The result is shown in FIG. 3. The obtained data show that resin concentrates troponin from blood samples (V) spiked with troponin, improving the detection compared with untreated whole blood (o). The library maintains the concentration differential between samples, with more troponin detected from samples with higher initial concentrations than from samples with lower initial concentrations.

### Example 5.

### Use of the combinatorial library for improved troponin detection by ELISA

Troponin (Biodesign, Cat #A86862H Lot # 3C07903) was spiked into human plasma (plasma spiked samples, ⋄) or human citrated blood before (blood pre-spiked samples, →) or after (blood post-spiked samples □)) incubation with library. The range of troponin concentration was from 0 to 100 ng/ml. Toyopearl AF Amino 650 M library (see Example 1), was swollen in DMF, washed with, and stored in 20% MeOH. The library resin was washed with PBS and equilibrated with citrate buffer directly before the experiments were performed. One ml of blood samples spiked (pre-spiked samples, A), or initially not spiked with Troponin (Troponin being added after elution of bound plasma proteins to the library, i.e. “post-spiked samples” , □) were flowed through a column containing library resin (100 ll of bed volume). The resin was washed three times with 1 ml of PBS to remove non-bound proteins. To remove extra PBS, columns were centrifuged for 4,000 g for 1 min in an Eppendorf tube. Bound proteins were eluted with 250vl of 0.05M HCl and immediately neutralized with 85 μl 0.5M NaH₂PO₄ pH 7.5. Eluates obtained from non-spiked blood samples were spiked with 0-100 ng/ml of Troponin (post-spiked samples, o).

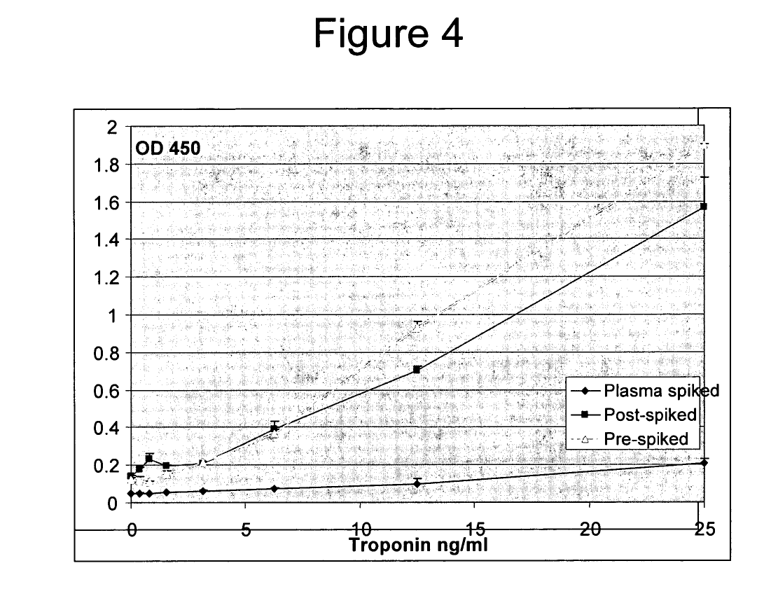

Evaluation of troponin in plasma and resin treated blood samples was performed by Troponin I ELISA, Bio-Quant kit (#BQ 015C) according to the manufacturer manual. The result is reflected in FIG. 4. The obtained data suggest that library-treatment significantly increases the sensitivity of troponin detection.

This specific graph demonstrates that blood plasma proteins recovered from the library and then spiked with Troponin (0) produces a more sensitive response than troponin spiked into whole plasma but not treated with the library (compare 0 and 0). This is probably because plasma proteins that interfere with the antibodies used in the ELISA have been decreased during treatment with the library.

### Example 6.

### Sequestration of highly interactive proteins

In order to improve the opportunities for trace proteins to bind to the library, the highly interactive protein fibrinogen was specifically sequestered from the rest of the plasma by performing the library incubation in the presence of a high-affinity ligand for fibrinogen.

Methods

100 μl of Toyopearl 650 M amino library (see Example 1) was swollen in diluted CPD (citrate, phosphate, dextrose buffer, Baxter Healthcare) and aliquoted into two BioRad columns, A and B. 200 μl of a fibrinogen-specific ligand, ARQFDF (see U.S. patent application Ser. No. 10/414,524), was similarly swollen and aliquoted into a third column (C). One ml of plasma diluted 1:4 with diluted CPD was added to columns A and C, and allowed to incubate, rotating, for 15 minutes. The resins and plasma from each was removed and placed into one half of two different wells of a 12-well microtiter plate into which a barrier made of Phenoseal caulk had been placed. This barrier divided the wells into two halves. The resin from column B was placed into the empty half of the well containing resin and plasma from column C (the ARQFDF) (well 1). The empty half of the well containing the contents of column A was left empty. 1 ml of CPD was added to the library: ARQFDF well to increase the solution volume sufficiently so that it freely passed over the barrier. This ensured that the plasma solution could contact both sets of resins while the resins remained separated and did not mix. The plate with the resins was incubated at room temperature, with gentle agitation, for 1 hour. Following incubation, all resins from the two wells were removed, unbound material from well 1 was saved, and the resins washed with CPD buffer. The proteins bound to the resins were visualized by elution and electrophoresis as described above in Example 1.

Results

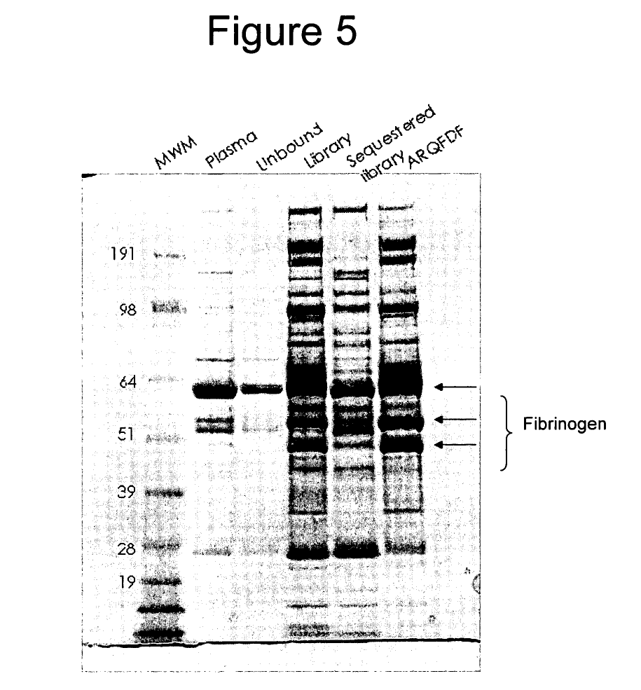

The results are presented in FIG. 5. There is a substantial difference in the pattern of proteins identified from library incubated with plasma in the presence of the specific ligand to the highly interactive fibrinogen (lane 5 (Sequestered library- from column B)) compared with the pattern of proteins identified from library incubated with plasma without the ligand specific for fibrinogen (Lane 4, library (column A)). There is a particular decrease in the amount of fibrinogen (arrows) bound by column B, and a concomitant enrichment of fibrinogen by the ARQFDF resin (Lane 6, ARQFDF (column C)). These data demonstrate that a promiscuously-binding protein can be specifically sequestered in the presence of ligand library, and that this improves the representation of other proteins to the library.

While the foregoing specification teaches the principles of the present invention, with examples provided for the purpose of illustration, it will be appreciated by one skilled in the art from reading this disclosure that various changes in form and detail can be made without departing from the true scope of the invention. All references cited are incorporated herein in their entirety.

What is claimed is:

1. A method of analyzing the amount of a target that binds to a ligand, which method comprises:
(i) providing one thousand or more different ligands, wherein each ligand is attached to a support to form one thousand or more ligand-support complexes
(ii) contacting the ligand-support complexes with two or more targets in a sample under conditions that allow at least one target to bind to at least two ligand-support complexes, thereby forming two or more target-ligand support complexes
(iii) separating non-bound targets from the target-ligand-support complexes
(iv) eluting at least a portion of the target of at least two target-ligand-support complexes in amounts dependent on the target's concentration in the starting sample
(v) detecting the target whereupon the relative amount of the target that binds to two or more ligand is analyzed.

2. The method of claim 1, wherein in step (i), ten thousand, one hundred thousand, one million or more different ligand -support complexes are provided.

3. The method of claim 1, wherein step (ii), more than two targets are provided.

4. The method of claim 3, wherein two or more targets compete for binding to one or more ligand-support complexes.

5. The method of claim 1, wherein the targets are in a biological fluid, food, environmental extract, or a composition comprising chemical compounds.

6. The method of claim 5, wherein the biological fluid is selected from the group consisting of whole blood, plasma, pooled plasma, intermediates from pooled plasma, biological products, serum, a cell homogenate, a conditioned medium, a fermentation broth, cerebrospinal fluid, urine, saliva, milk, ductal fluid, tears, perspiration, lymph and semen.

7. The method of claim 6, wherein the biological fluid is from a host afflicted with a disease.

8. The method of claim 1, wherein the environmental sample or extract is selected from the group consisting of soil, an extract from a naturally occurring body of water, food, a sample from air and a swab from a building.

9. The method of claim 1, wherein the targets are selected from the group consisting of cells, bacteria, viruses, yeast, microparticles in blood, proteins, peptides, amino acids, nucleic acids, carbohydrates, lipids, drugs, synthetic inorganic compounds, synthetic organic compounds, isoforms of any of the foregoing, and combinations of any of the foregoing.

10. The method of claim 1, wherein the targets are liberated into the biological fluid as a result of tissue damage.

11. The method of claim 10, wherein the target is troponin.

12. The method of claim 10, wherein the target is a virus.

13. The method of claim 12, wherein the virus is a parvovirus.

14. The method of claim 1, wherein the ligands are organic molecules.

15. The method of claim 14, wherein the organic molecules are selected from the group consisting of amino acids, peptides nucleic acids, carbohydrates, sugars, lipids, steroids, drugs, vitamins, and cofactors.

16. The method of claim 15, wherein the peptides consist essentially of about 1-15 amino acids.

17. The method of claim 1, wherein the support is a resin bead.

18. The method of claim 17, wherein the resin bead comprises a material selected from a group consisting of agarose, ethylene glycol, fluoropolymers, dimethacrylate, glycidol methacrylate, polyacrylate, polyesters, polyethylene glycol, polyhydroxymethacrylate, dextran, cellulose, polypropylene, polyethylene oxides, polysaccharide derivatives, of any of the foregoing, and combinations of the foregoing.

19. The method of claim 18, wherein the resin is ToyoPearl 650M,

20. The method of claim 1, wherein step (iii) is performed by washing with saline.

21. The method of claim 1, wherein step (iv) is carried out in a medium containing a competitive binding agent, which binds to the target of at least one target-ligand-support complex, thereby causing the ligand to dissociate from at least a portion of the target.

22. The method of claim 1, wherein two or more target-ligands-support complexes are sub-pooled before eluting at least a portion of the target of at least two target-ligand-support complex from one subpool in an amount proportional to their presence in the starting sample, then detecting the at least one target whereupon the target that binds to two or more ligand-support complexes is analyzed.

23. The method of claim 22, wherein the target-ligand-support complexes are separated into sub-pools by means of a semi-permeable membrane.

24. The method of claim 23, wherein the targets are separated into sub-pools by equilibrium affinity dialysis using target specific affinity resins comprising ligands.

25. The method of claim 24, wherein the specific affinity resins comprising ligands bind to the most interactive proteins present in the sample containing the two or more targets.

26. The method of claim 25, wherein the most interactive proteins present in the sample are selected from the group consisting of immunoglobulin, fibrinogen, HDL, and LDL.

27. The method of claim 22, wherein the target ligand support complexes and the affinity resins are separated into sub-pools by physical separation of the affinity resins by a magnetic field, sedimentation rate, density or size.

28. The method of claim 1, wherein step (v) comprises performing mass spectrometry to detect the target.

29. The method of claim 1, wherein step (v) comprises performing gel-electrophoresis to detect the target.

30. The method of claim 1, wherein step (v) comprises performing an enzyme assay to detect the target.

31. The method of claim 1, wherein step (v) comprises performing an immunological assay to detect the target.

32. The method of claim 31, wherein the immunological assay is selected from the group consisting of an ELISA, nephelometry assay, and Western blot based assay.

33. The method of claim 1, wherein step (v) comprises contacting cells with the eluted sample obtained in step (v) and detecting a cellular response.

34. The method of claim 33, wherein the cellular response is cell death, growth or differentiation.

35. The method of claim 1, wherein a plurality of targets are initially present at different amounts and have a first concentration range and variance, and the eluted captured targets have a second concentration range and variance, wherein the amounts of each of the different binding ligands are selected to capture amounts of the different targets such that the second variance is less than the first variance.

36. A method for detecting diagnostic biomarkers in a species or tissue comprising
(i) providing a first biological sample from a subject having a first phenotype and a first plurality of different targets
(ii) providing a second biological sample from a second subject having a second phenotype with a second plurality of different targets
(iii) treating separately the first and the second plurality of different targets according to the method of claim 1, thereby creating a third and a fourth set of biological samples
(iv) identifying at least one target that is differentially present in the third and fourth set of biological samples, whereby the at least one target species and its approximate concentration is a biomarker for distinguishing the first phenotype from the second phenotype.

37. A method for reducing the relative amounts of targets in a sample, the method comprising the steps of:
(i) providing a first sample comprising a first plurality of different targets having a first variance in amounts;
(ii) contacting the first sample with a plurality of different binding ligands, each binding ligand present in a determined amount
(iii) binding a portion of the first different targets from the first sample to the different binding ligands and removing unbound targets from the newly formed target-ligand-support complexes; and
(iv) eluting the bound targets from the target-ligand-support complexes to produce a second sample comprising a second plurality of different targets whereby the variance in amounts of targets in the second sample is less than the variance in amounts of targets in the first sample.
